SEQUENCE LISTING (21 sequences)

SEQ ID NO: 1 - DNA, 16 bp, Artificial Sequence
  not from a natural source.  sequence used as an example only.
atcgtctagcgcgaat

SEQ ID NO: 2 - DNA, 16 bp, Artificial Sequence
  not from a natural source.  sequence used as an example only.
tagcagatcgcgctta

SEQ ID NO: 3 - DNA, 16 bp, Artificial Sequence
  Artificial sequences not from any particular source.  Sequences used for example only.
atugtutagugugaat

SEQ ID NO: 4 - DNA, 16 bp, Artificial Sequence
  not from a natural source.  sequence used as an example only.
taguagatugugutta

SEQ ID NO: 5 - DNA, 16 bp, Artificial Sequence
  not from a natural source.  sequence used as an example only.
atcgtutagcgcgaat

SEQ ID NO: 6 - DNA, 16 bp, Artificial Sequence
  not from a natural source.  sequence used as an example only.
tagcagatugcgctta

SEQ ID NO: 7 - DNA, 16 bp, Artificial Sequence
  not from a natural source.  sequence used as an example only.
attgtttagtgtgaat

SEQ ID NO: 8 - DNA, 16 bp, Artificial Sequence
  not from a natural source.  sequence used as an example only.
taacaaatcacactta

SEQ ID NO: 9 - DNA, 16 bp, Artificial Sequence
  not from a natural source.  sequence used as an example only.
atcatctaacacaaat

SEQ ID NO: 10 - DNA, 16 bp, Artificial Sequence
  not from a natural source.  sequence used as an example only.
tagtagattgtgttta

SEQ ID NO: 11 - DNA, 16 bp, Artificial Sequence
  not from a natural source.  sequence used as an example only.
atcgtttagcgcgaat

SEQ ID NO: 12 - DNA, 16 bp, Artificial Sequence
  not from a natural source.  sequence used as an example only.
tagcaaatcgcgctta

SEQ ID NO: 13 - DNA, 16 bp, Artificial Sequence
  not from a natural source.  sequence used as an example only.
atcgtctaacgcgaat

SEQ ID NO: 14 - DNA, 16 bp, Artificial Sequence
  not from a natural source.  sequence used as an example only.
tagcagattgcgctta

SEQ ID NO: 15 - DNA, 71 bp, Artificial Sequence
  not from a natural source.  sequence used as an example only.
attgaaggctgccaaaatcgggtccctcctgtcgggagtatctccctcctcccaaacttcacttgagactc

SEQ ID NO: 16 - DNA, 71 bp, Artificial Sequence
  not from a natural source.  sequence used as an example only.
attgaaggttgttaaaattgggttttttttgttgggagtatctttttttttttaaattttatttgagattt

SEQ ID NO: 17 - DNA, 71 bp, Artificial Sequence
  not from a natural source.  sequence used as an example only.
attgaaggttgttaaaatcgggttttttttgtcgggagtatttttttttttttaaattttacttgagattt

SEQ ID NO: 18 - DNA, 71 bp, Artificial Sequence
  not from a natural source.  sequence used as an example only.
attgaaggttgttaaaatcgggttttttttgtcgggagtatttttttttttttaaattttatttgagattt

SEQ ID NO: 19 - DNA, 71 bp, Artificial Sequence
  not from a natural source.  sequence used as an example only.
attgaaggttgttaaaattgggttttttttgtcgggagtatttttttttttttaaattttatttgagattt

SEQ ID NO: 20 - DNA, 71 bp, Artificial Sequence
  not from a natural source.  sequence used as an example only.
attgaaggttgttaaaatcgggttttttttgttgggagtatttttttttttttaaattttatttgagattt

SEQ ID NO: 21 - DNA, 71 bp, Artificial Sequence
  not from a natural source.  sequence used as an example only.
attgaaggttgttaaaattgggttttttttgttgggagtatttttttttttttaaattttatttgagattt

(19) United States
(12) Patent Application Publication
(10) Pub. No.: US 2015/0141256 A1
(43) Pub. Date: May 21, 2015

(54) COMPOSITIONS AND METHODS FOR BISULFITE CONVERTED SEQUENCE CAPTURE

(71) Applicant: Roche NimbleGen, Inc., Madison, WI (US)

(72) Inventors: Daniel Burgess, Madison, WI (US); Jeffrey Jeddeloh, Madison, WI (US)

(21) Appl. No.: 14/338,748

(22) Filed: Jul. 23, 2014

Related U.S. Application Data
(60) Provisional application No. 61/859,406, filed on Jul. 29, 2013.

(51) Int. Cl. C12Q 1/68

(52) U.S. Cl. CPC C12Q 1/6876; C12Q 2600/154
USPC 506/2; 506/16

(57) ABSTRACT

This invention relates generally to composition and methods for characterizing a methylome which comprises all or substantially all methylation states of a genome. In particular, a plurality of oligonucleotides, each representing nearly every possible methylation state of the cytosine position of each CG dinucleotide pair within a target nucleic acid of interest, and methods of using the plurality are provided herein.

### RELATED APPLICATIONS

This application claims the benefit of U.S. Provisional Application No. 61/859,406 filed Jul. 29, 2013, the disclosure of which is hereby incorporated by reference in its entirety.

### FIELD OF THE INVENTION

This invention relates generally to composition and methods for characterizing a methylome which comprises all or substantially all methylation states of a genome. In particular, the present invention relates to a plurality of oligonuclotides and methods of using the plurality to identify the methylation state of the cytosine position of each CG dinucleotide pair of a target nucleic acid of interest.

### BACKGROUND OF THE INVENTION

The gold standard protocol for characterizing post-replication methylation of cytosines positioned adjacent to a guanine in a cytosine-guanine (CG) dinucleotide pair is bisulfite conversion followed by DNA sequencing. The methylation state of the cytosine position of each CG dinucleotide pair within a nucleic acid of interest will vary according to the molecule's sequence and can exist at any level between 0% methylated (i.e., all such cytosines are sensitive to bisulfite treatment) and 100% methylated (i.e., none of such cytosines are sensitive to bisulfite treatment). Thus, across a eukaryotic genome (e.g., human genome), the vast number of potential methylation states can be staggering. To identify all methylation occupancies in a genomic DNA sample, the set of oligonucleotides hybridizable to the bisulfite converted genomic DNA would have to represent each and every potential methylation states.

There remains a need in the art for methods capable of characterizing DNA methylation patterns with single-base resolution but on a genome-wide scale. There also remains a need for methods of identifying methylation occupancies on a genome-wide scale that are also capable of discriminating single nucleotide polymorphisms from unmethylated bases.

### BRIEF DESCRIPTION OF THE DRAWINGS

FIG. 1 demonstrates an embodiment of the workflow used in the present invention.

FIG. 2 demonstrates the bisulfite conversion of DNA.

FIG. 3 depicts the wobble-probe strategy.

FIG. 4 shows a comparison of methylation data using whole genome sequencing (FIG. 4A) versus data obtained using the capture pools and protocol of the present invention (FIG. 4B).

FIG. 5 shows results of application of the method of the invention to a Burkitt lymphoma cell line. The depth of coverage metrics are listed in the table in FIG. 5A, with the reproducibility (r-squared of methylation ratios) demonstrated in FIG. 5B.

### DETAILED DESCRIPTION OF THE INVENTION

The present invention is directed to methods and compositions for genome-wide mapping of the methylation state of an organism's whole genome, or an organism's “methylome.” The present invention is based, at least in part, on the Inventors' discovery of methods for generating a plurality of oligonucleotides, each representing nearly every possible methylation state of the cytosine position of each CG dinucleotide pair within a target sequence of interest. CG dinucleotides are not uniformly distributed throughout the genome, but are concentrated in regions of repetitive genomic sequences and in CpG “islands,” which are commonly associated with gene promoters. The Inventor's discovery and the invention provided herein are particularly important given that it has not been technically or economically feasible using conventional probe synthesis protocols to obtain a plurality of oligonucleotides that is sufficiently comprehensive to characterize all or nearly all of the possible methylation sites in a long bisulfite converted nucleic acid sample, including bisulfite converted nucleic acid samples as large as a eukaryotic genome.

Compositions

Accordingly, in one aspect, the present invention provides a plurality of oligonucleotides. In preferred embodiments, oligonucleotides of the plurality are hybridizable to at least a portion of a bisulfite converted genomic nucleic acid sample of a target organism. In some cases, each oligonucleotide comprises a wobble base at each cytosine-complementary position of a dinucleotide pair complementary to a cytosine-guanine dinucleotide pair in an unconverted genomic nucleic acid of the target organism. As used herein, the term “wobble base” refers to alternative bases incorporated into an oligonucleotide at a particular position when synthesized in the presence of a known equimolar mixture of two or more deoxynucleoside triphosphates (dNTPs) (e.g., an equimolar mixture of dNTPs comprising deoxycytidine triphosphate (dCTP) and deoxythymidine triphosphate (dTTP)).

Oligonucleotides of the plurality provided herein can be a double-stranded or single-stranded oligonucleotide. Preferably, oligonucleotides of the plurality are support-immobilized. For example, oligonucleotides of the present invention can be synthesized on a substrate (e.g., solid support) using, for example, a maskless array synthesizer (MAS) (described in U.S. Pat. No. 6,375,903). MAS provides for in situ synthesis of oligonucleotide sequences directly on a solid substrate. Accordingly, nascent oligonucleotides are support-immobilized. In general, MAS-based oligonucleotide synthesis technology allows for the parallel synthesis of over 4 million unique oligonucleotides in a very small area of a standard microscope slide.

In some cases, an oligonucleotide can further comprise an adapter sequence. Adapter sequences are located at either or both ends of the oligonucleotide. In some cases, an adapter sequence can comprise biotin. In other cases, an adapter sequence can comprise a fluorophore. Preferably, adapter sequences can be configured for purification and amplification and for sequencing applications.

In another aspect, the present invention provides a hybridization array comprising a plurality of features. As used herein, “feature” and “features” refer to specific locations on an array at which oligonucleotides are synthesized. In some cases, one nucleotide sequence is synthesized at each feature of the array (i.e., multiple probes can be synthesized in each feature, but all probes at the feature have the same nucleotide sequence). In other cases, oligonucleotides of different sequences can be present within one feature of the array. The ratio and direction (5′-3′, or 3′-5′) of these oligonucleotides can be controlled.

In a preferred embodiment, a maskless array synthesizer (MAS) provides for in situ synthesis of oligonucleotide sequences directly on a solid substrate. In general, MAS-based oligonucleotide microarray synthesis technology allows for parallel oligonucleotide synthesis at millions of unique oligonucleotide features on a solid substrate such as a glass microscope slide.

Where it is desirable to obtain oligonucleotides for the Watson (forward, non-complementary) and Crick (reverse, complementary) strands of a bisulfite converted target nucleic acid, one or more of the following oligonucleotide design protocols can be used. To generate oligonucleotides for a fully unmethylated sample, all cytosines of each oligonucleotide are changed to thymines. To generate oligonucleotides for a fully methylated sample, all cytosines except those positioned in a CG dinucleotide pair are changed to thymines. For oligonucleotides hybridizable to a non-complementary reverse strand, cytosines are modified as described above and, subsequently, each oligonucleotide sequence is reverse-complemented back to an original Crick strand. For wobble base incorporation, all cytosines not adjacent to a guanine are modified to thymine and each instance of a CG dinucleotide pair is replaced with a “YG” dinucleotide pair, where Y represents the International Union of Pure and Applied Chemistry (IUPAC) code for either a cytosine or a thymine/uracil at that position. The IUPAC code is a 16-character code which allows the ambiguous specification of nucleic acids. The code can represent states that include single specifications for nucleic acids (A, G, C, T/U) or allows for ambiguity among 2, 3, or 4 possible nucleic acid states.

The compositions provided herein are useful for, for example, identifying methylated bases of a bisulfite converted target nucleic acid. Under acidic conditions, sodium bisulfite preferentially deaminates cytosine to uracil in a nucleophilic attack while the methyl group on 5-methylcytosine protects the amino group from the deamination. As a result, methylated cytosine is not converted under these conditions. Accordingly, an original methylation state can be analyzed by sequencing bisulfite converted DNA (e.g., bisulfite converted genomic DNA) and comparing the cytosine position of each cytosine-guanine (CG) dinucleotide pair of an unconverted nucleic acid to bases at the corresponding positions in the sequence of a bisulfite converted nucleic acid of interest. When compared to an unconverted genomic nucleic acid of a target organism, cytosine bases remaining in the interrogated bisulfite converted DNA sample of the target organism are indicative of methylated cytosines in the genome.

In preferred embodiments, sodium bisulfite is used to convert an unmethylated cytosine base of a cytosine-guanine (CG) dinucleotide pair to a uracil. Sodium bisulfite can be a mixture of NaHSO₃ and Na₂S₂O₅. In some cases, magnesium sulfite can be used for bisulfite conversion. In some cases, other chemical compounds can be used to convert cytosines to uracil. For example, a nucleophilic organo-sulfur compound (e.g., X₂—S(O)—X₁, where X is methanol, ethanol, or (CH₂)₅) can be used in place of bisulfite. Suitable mono-substituted sulfur nucleophiles include, without limitation, sulphurous acid monomethyl esters (e.g., monomethyl sulfite), methyl sodium sulfite, phenyl hydrogen sulfite, sodium phenyl sulfite, methylsulfinic acid or ethylsulfinic acid. Other possible substances can include bis-substituted sulfur nucleophiles such as sulphurous acid dimethyl ester, methanesulfinylmethane, 2-methyl-propane-2sulfinic acid diethylamide, [1,3,2]dioxathiolane 2-oxide, and 2,5-diethyl [1,2,5]thiadiazolidine 1-oxide.

Any appropriate bisulfite conversion protocol can be used. Preferably, bisulfite conversion is performed using highly pure (e.g., phenol-chloroform extracted) nucleic acids. Optionally, a desulphonation step is performed following bisulfite conversion of a nucleic acid sample. In some cases, a bisulfite converted nucleic acid sample can be purified for subsequent use. Any appropriate method can be used to purify a bisulfite converted nucleic acid sample. Several conventional methods of DNA purification are known by those practicing in the art.

In a preferred embodiment, bisulfite converted, purified DNA is subsequently amplified by, for example, polymerase chain reaction (PCR) using specific primers in which uracil corresponds to thymine according to rules of nucleotide base-pairing. Any appropriate downstream detection technique can be performed using amplified bisulfite converted DNA. For example, any appropriate sequencing or microarray detection method(s) can be performed.

Strands of a bisulfite converted nucleic acid sample are no longer complementary. In some cases, it may be useful to amplify and analyze each strand of a bisulfite converted nucleic acid using, for example, strand-specific PCR primers and PCR. Accordingly, strand-specific primers can be designed to amplify, clone, and sequence the individual strands (e.g., sense and antisense) to determine the methylation patterns of each. Due to de novo methylation of the nascent strand by methyltransferase, methylation patterns of the sense and antisense strands should be identical. In a preferred embodiment, however, oligonucleotides provided herein have the ability to recover both strands of bisulfite converted nucleic acid in order to discriminate between a single nucleotide polymorphism (SNP) and an unmethylated base. In particular, oligonucleotides provided herein can be used to distinguish between a thymine SNP in a bisulfite converted target nucleic acid and an unmethylated site by identifying A or G bases at the corresponding position in the complement strand. Where this corresponding position in the complement strand is a guanine (G), that position is identified as being unmethylated. The presence of an adenosine (A) in the corresponding position in the complement strand indicates the presence of a SNP in the target nucleic acid of interest. Such analysis requires capture of the sense and antisense strands from a bisulfite treated target nucleic acid.

In some cases, a catalyst of the bisulfite conversion reaction is used. For example, in some cases, a polyamine such as Tetraethylenepentaminepenta-hydrochloride (TETRAEN) can be used to catalyze the bisulfite conversion of cytosine to uracil. The amine salt TETRAEN comprises five catalytic amine groups, each of which harbors opposite charges which drive electrons in the cytosine to the pyrimidine ring where the bisulfite reaction occurs. Other reaction catalyzing polyamines useful for the methods provided herein can include, without limitation, diamines, triamines (e.g., diethylene triamine (DETA)), guanidine, tetramethyl guanidine, tetraamines, and other compounds containing two or more amine groups, and salts thereof.

Methods

In a further aspect, the present invention provides methods of identifying a methylation state of the cytosine position of a cytosine-guanine (CG) dinucleotide pair in a target nucleic acid molecule. For example, the present invention provides a method for identifying methylated bases within a bisulfite converted target nucleic acid sequence. Bisulfite conversion and sequencing provide detailed information of the methylation pattern of a nucleic acid of a target organism with single-base resolution. Bisulfite sequencing exploits the preferential deamination of cytosine bases to uracil bases in the presence of sodium hydroxide (NaOH) and sodium bisulfite. Methylated cytosine bases (5-methylcytosine), if present, are found almost exclusively at the cytosine position of a CG dinucleotide pair (e.g, 5′-CG-3′). Under acidic conditions, sodium bisulfite preferentially deaminates cytosine to uracil in a nucleophilic attack while the methyl group on 5-methylcytosine protects the amino group from the deamination. As a result, methylated cytosine is not converted under these conditions. Accordingly, the DNA's original methylation state can be analyzed by sequencing the bisulfite converted DNA and comparing the cytosine position of each cytosine-guanine (CG) dinucleotide pair of an unconverted nucleic acid to bases at the corresponding positions in the sequence of a bisulfite converted nucleic acid of interest. The cytosine position of a cytosine-guanine (CG) dinucleotide pair of the unconverted nucleic acid is identified as having been unmethylated if the corresponding position in the sequence of a bisulfite converted nucleic acid of interest is now occupied by thymine. The cytosine position of a cytosine-guanine (CG) dinucleotide pair of the unconverted nucleic acid is identified as having been methylated if the corresponding position in the sequence of a bisulfite converted nucleic acid of interest is occupied by cytosine.

In a preferred embodiment, a method according to the present invention can comprise contacting a plurality of oligonucleotides to a bisulfite converted nucleic acid of a target organism. Preferably, the bisulfite converted nucleic acid is from a genomic DNA sample. For example, each oligonucleotide can be hybridizable to at least a portion of a bisulfite converted genomic nucleic acid sample of a target organism. Also, each oligonucleotide comprises a wobble base at each position of a dinucleotide pair complementary to a cytosine-guanine (CG) dinucleotide pair in an unconverted genomic nucleic acid of the target organism. As a result of contacting oligonucleotides to a bisulfite converted nucleic acid sample as described herein, bisulfite converted target nucleic acid molecules can be captured in hybridization complexes with at least a subset of the plurality of oligonucleotides.

In a preferred embodiment, a method of the present invention further comprises providing bisulfite converted C0t1 DNA as a blocking reagent. Providing bisulfite converted C0t1 DNA can improve efficacy and specificity of a method provided herein. Preferably, the blocking reagent is bisulfite converted C0t1 DNA from the same species as the target nucleic acid sequence of interest. For example, if the bisulfite converted target nucleic acid is from a human, bisulfite converted human C0t1 DNA can be provided as a blocking reagent. In some cases, therefore, a method provided herein can comprise contacting a plurality of oligonucleotides to a bisulfite converted nucleic acid of a target organism in the presence of bisulfite converted C0t1 DNA. In some cases, the bisulfite converted C0t1 DNA and the target organism are of the same species.

A method according to the present invention can further comprise the steps of (i) separating hybridization complexes from unbound and non-specifically bound nucleic acid molecules, and (ii) eluting captured bisulfite converted target nucleic acid molecules from the hybridization complexes.

A method according to the present invention also can comprise sequencing eluted bisulfite converted target nucleic acid sequences. Any appropriate DNA sequencing method can be used according to the methods provided herein. Upon sequencing, methylated bases of an eluted bisulfite converted target nucleic acid sequence can be identified. The identifying step can comprise comparing unconverted genomic nucleic acid of the target organism to the eluted bisulfite converted target nucleic acid sequence, as above.

Unless defined otherwise, all technical and scientific terms used herein have the same meaning as commonly understood by one of ordinary skill in the art to which the invention pertains. Although any methods and materials similar to or equivalent to those described herein can be used in the practice or testing of the present invention, the preferred methods and materials are described herein.

The invention will be more fully understood upon consideration of the following non-limiting Examples. All papers and patents disclosed herein are hereby incorporated by reference as if set forth in their entirety.

### Examples

DNA methylation has been shown to have a role in a host of biological processes, including silencing of transposable elements, stem cell differentiation, embryonic development, genomic imprinting, and inflammation, as well as many diseases, including cancer, cardiovascular disease, and neurologic diseases. Epigenetic modifications can also affect drug efficacy by modulating the expression of genes involved in the metabolism and distribution of drugs, as well as the expression of drug targets, contributing to variability in drug responses among individuals. There are currently a number of tools to study DNA methylation status, either at a single locus level, using methods like methylation-specific PCR or MALDI-TOF-MS, or at a broader, genome-wide level, using DNA microarrays, reduced representation bisulfite sequencing (RRBS), or whole genome shotgun bisulfite sequencing. The latter method is preferred by many researchers, as it provides DNA methylation status at base pair resolution and allows for the assessment of percent methylation at each position in the genome. However, it is expensive, in terms of money and analysis, to generate such data for the entire genome, when generally only a subset of the genome is of interest to most researchers. In one embodiment, this invention is a system for the targeted enrichment of bisulfite treated DNA, allowing researchers to focus on a subset of the genome for high resolution methylation analysis. Regions ranging in size from 10 kb to 75 Mbp may be targeted, and multiple samples may be multiplexed and sequenced together to provide an inexpensive method of generating methylation data for a large number of samples in a high throughput fashion.

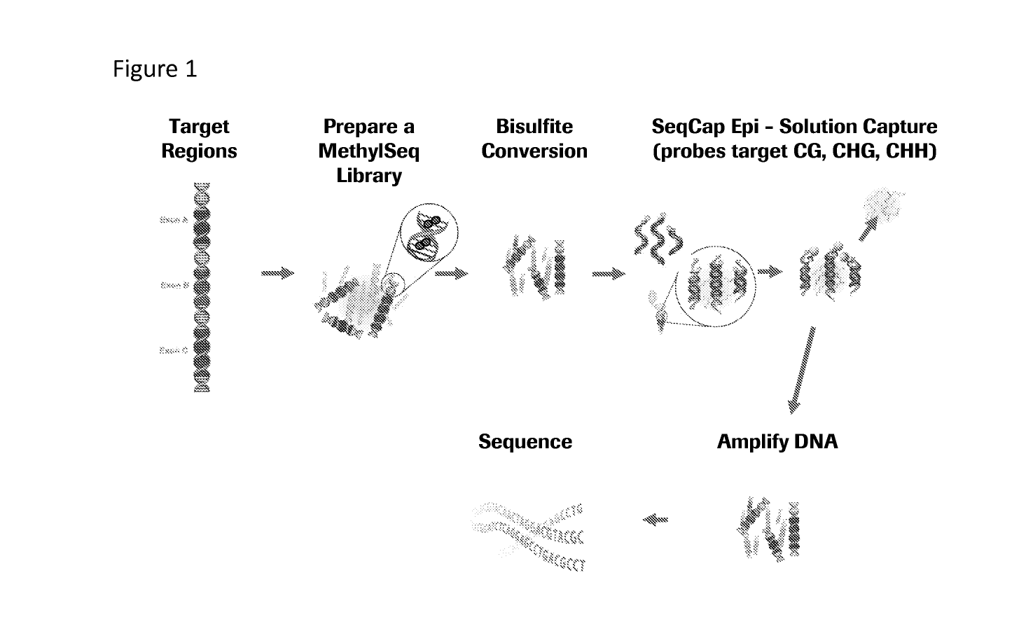

FIG. 1 demonstrates an embodiment of the workflow used in the present invention. Unlike most sequence capture protocols (e.g., standard SeqCap EZ protocols from Nimblegen), the process of the present invention begins with bisulfite converted genomic DNA. In FIG. 1, the researcher determines the appropriate target regions for methylation studies, as opposed to examination of the whole genome in certain standard methylation study applications. The genomic sample is fragmented, bisulfite converted and a library is generated with methylated sequencing adapters, or the library may be generated with methylated sequence adapters prior to bisulfite conversion. The samples are then amplified for several cycles using the sequencing adapters, generally from 4-8 cycles. Sequence capture is then performed by hybridizing a wobble pool of biotinylated probes to the converted genomic regions of interest (i.e., hybridize, streptavidin-biotin capture, and wash to remove non-specifically bound material and perform LM-PCR for 10-18 cycles). In some embodiments, it is useful to employ a bisulfite converted Cot1 DNA form the species of interest as a blocking agent (e.g., if the sample is human, the bisulfite converted blocking agent is human Cot1 DNA). Further, certain embodiments may also employ “blocking oligos” complementary to library adapters, designed to suppress cross-hybridization among library adapters and thus increase enrichment specificity. The captured targets are generally amplified, and then are sequenced. The bisulfite converted reads are mapped (i.e., aligned to the reference sequence or assembled de novo), and the methylation status determined.

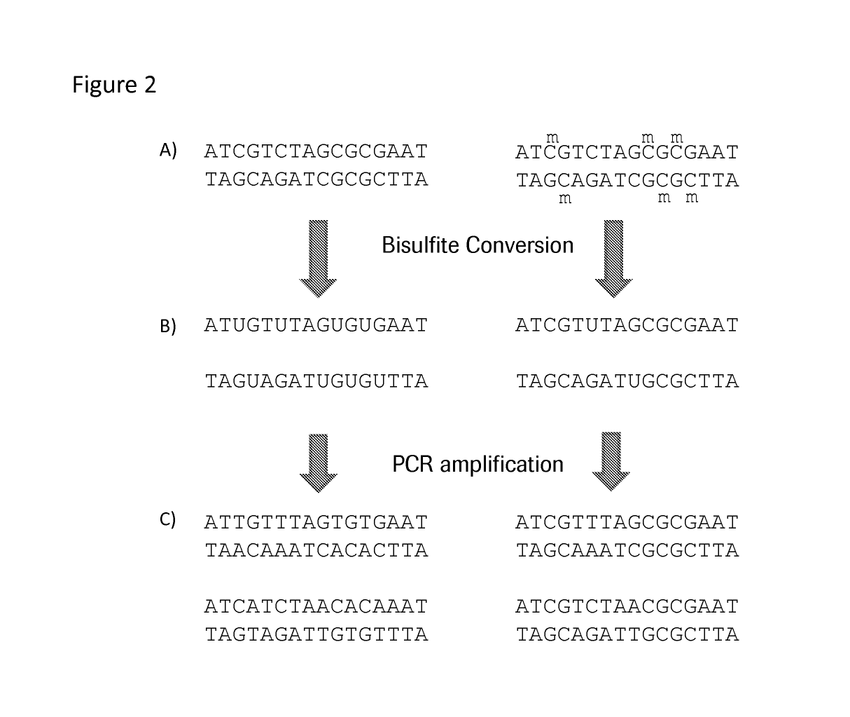

FIG. 2 demonstrates the bisulfite conversion of DNA. Cytosines next to guanine may be methylated (m) in the genome. FIG. 2 represents identical sequences in which none of the cytosines are methylated (left column, FIG. 2A) versus the same sequences which are partially methylated (right column, FIG. 2A). The genomic samples are subjected to bisulfite conversion, wherein unmethylated cytosines are converted to uracil, while methylated cytosines remain unchanged. The unmethylated cytosines, once converted to uracil, act as thymine for purposes of DNA pairing. The strands are then PCR amplified. After PCR amplificatioin, the strands are no longer complimentary. Bisulfite treatment effectively doubles the size of the genome, because the forward and reverse strands are no longer complementary. The partial conversion of C's to T's also complicates probe design and analysis.

Methylation varies by tissue, by condition, and by time. For a short sequence with 3 possible methylation sites, there are 32 possible short fragments that could be produced:

| Combination(s) | |
|---|---|
| 1 | 0 methylated C's × 2 bisulfite-treated fragments × |
| | 2 additional strands created by PCR = 4 |
| 3 | 1 methylated C's × 2 bisulfite-treated fragments × |
| | 2 additional strands created by PCR = 12 |
| 3 | 2 methylated C's × 2 bisulfite-treated fragments × |
| | 2 additional strands created by PCR = 12 |
| 1 | 3 methylated C's × 2 bisulfite-treated fragments × |
| | 2 additional strands created by PCR = 4 |

Thus, bisulfate treatment leads to significant sample complexity. Thus, targeted enrichment is of great benefit when examining the methylation state of a particular region of interest.

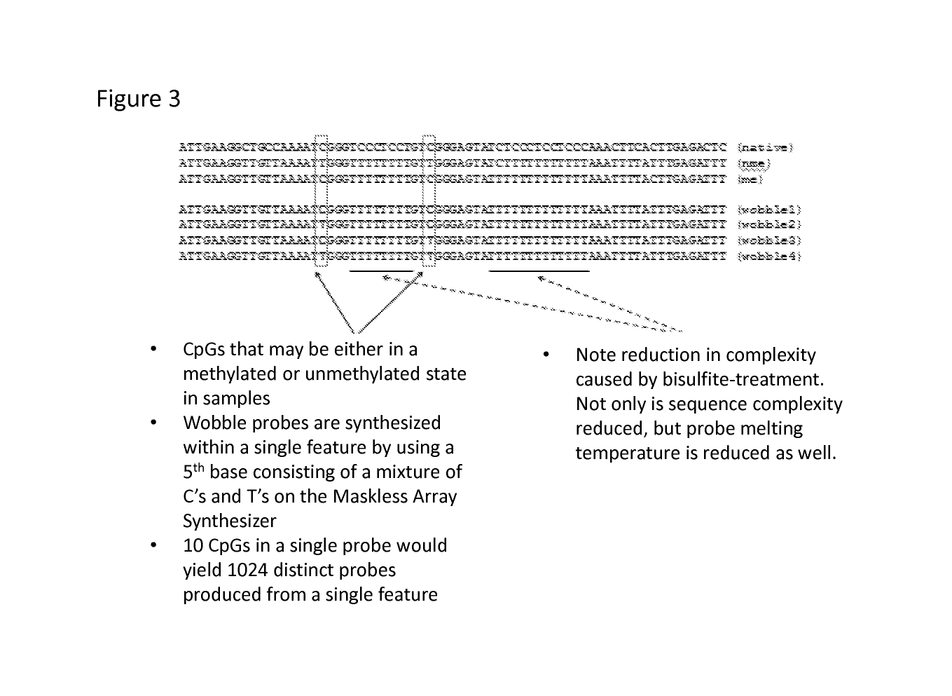

The most interesting areas of the genome to look at are those that exhibit differential methylation. Those regions may be hyper-methylated, partially methylated or hypo-methylated. Thus capture probes must be able to hybridize to a range of bisulfite-converted molecules from any given region. The present invention employs a strategy to design 3 sets of capture probes: One set of probes (me) against fragments where all CpG's are assumed to be methylated, and thus preserved after bisulfite-treatment. A second set of probes (nme) against fragments where all CpG's are assumed to be un-methylated, and thus all C's are converted to T's. The final set of probes (wobble) is designed to capture the remaining fragments where 1 or more CpG's are methylated. FIG. 3 depicts this strategy, demonstrating the native sequence for a particular region, the nme probe (wherein the cytosines in the CpG islands are converted), the me probe (wherein the cytosines remain unchanged), and the wobble pool consisting of all possible combinations. Wobble probes are synthesized within a single feature by using a “5^{th} base” consisting of a mixture of cytosine and thymine during the synthesis, preferably using Maskless Array Synthesis. 10 CpGs in a single probe would yield 2¹⁰=1024 distinct probes produced from a single feature.

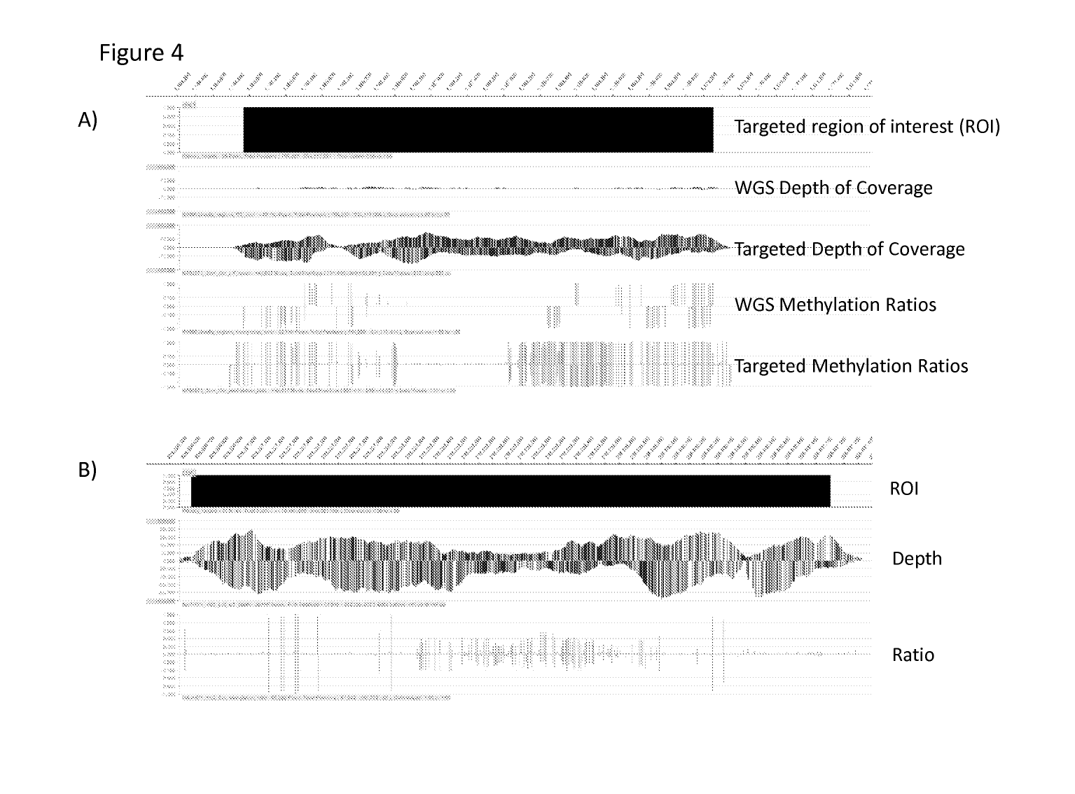

FIG. 4 shows a comparison of methylation data using whole genome sequencing (FIG. 4A) versus data obtained using the capture pools and protocol of the present invention (FIG. 4B). The figure shows data taken from a bivalent domain as the targeted region of interest, showing a region of hypo-methylation flanked by hyper-methylated regions. In this figure, the depth of coverage tracks are scaled to the same height. Whole Genome Shotgun (WGS) bisulfite sequencing provides low depth of coverage, making in nearly impossible to recognize this pattern of methylation. On the other hand, the method of the present invention, using wobble probes, provided increased depth of coverage. This increased depth allowed reliable determination of intermediate methylation states.

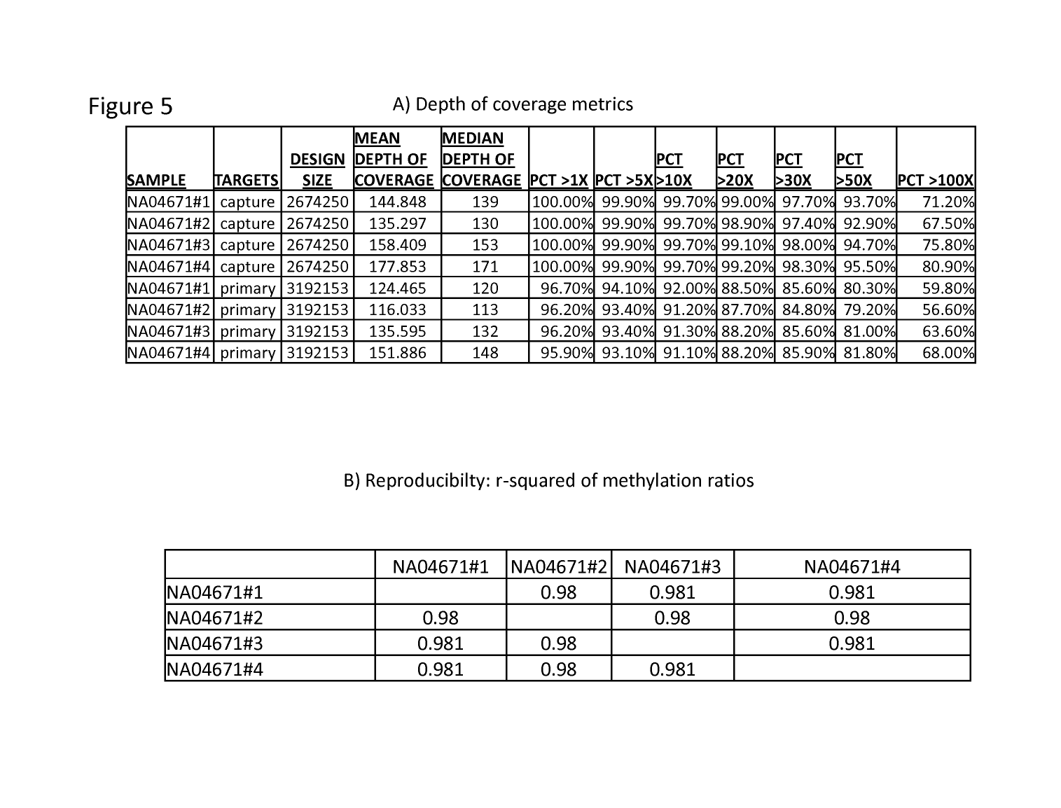

NA04671, a Burkitt lymphoma cell line, was subjected to targeted enrichment using the bisulfite sequencing methods of the present invention. The depth of coverage metrics are listed in the table in FIG. 5A, with the reproducibility (r-squared of methylation ratios) demonstrated in FIG. 5B. The capture targets (where probes could be designed) cover 93% of the primary targets (the regions of interest). Each sequencing run utilized approximately ⅓ of a MiSeq sequencer lane (2×100 bp). Normal recommended depth of coverage for whole genome shotgun bisulfite-sequencing is 30× (15× for each strand), or at least 2-3 lanes of HiSeq 2000 (2s×100 bp).

Capture and sequencing using the present invention provides a method of examining methylation states at unprecedented levels. By specifically targeting regions of interest, the resources devoted to sequencing are greatly reduced, allowing multiple samples to be multiplexed together and/or providing much higher depth of coverage per sample. The increased depth of coverage enables fractional changes in methylation states to be determined, providing a means to discover regions of differential methylation at high sensitivity.

We claim:

1. A plurality of oligonucleotides hybridizable to at least a portion of a bisulfite converted genomic nucleic acid sample of a target organism, each oligonucleotide comprising, at each position of a dinucleotide pair complementary to a cytosine-guanine dinucleotide pair in an unconverted genomic nucleic acid of the target organism, a wobble base at each cytosine-complementary position.

2. The oligonucleotides of claim 1, wherein the wobble base is incorporated during oligonucleotide synthesis using an equimolar mixture of nucleoside triphosphates (dNTPs).

3. The oligonucleotides of claim 2, wherein the equimolar mixture of dNTPs comprises deoxycytidine triphosphate (dCTP) and deoxythymidine triphosphate (dTTP).

4. The oligonucleotides of claim 3, wherein the equimolar mixture of dNTPs further comprises deoxyadenosine triphosphate (dATP), deoxyguanosine triphosphate (dGTP), or deoxyuridine triphosphate (dUTP).

5. The oligonucleotides of claim 1, wherein each oligonucleotide further comprises an adapter sequence at either or both ends of the oligonucleotide.

6. The oligonucleotides of claim 5, wherein the adapter sequence at either or both ends of the oligonucleotide comprises biotin.

7. The oligonucleotides of claim 5, wherein the adapter sequence at either or both ends of the oligonucleotide comprises a fluorophore.

8. The oligonucleotides of claim 1, wherein the oligonucleotides are support-immobilized.

9. The oligonucleotides of claim 1, wherein at least a subset of the oligonucleotides are capable of discriminating a thymine single nucleotide polymorphism (SNP) from an unmethylated cytosine in the unconverted genomic nucleic acid.

10. A hybridization array comprising a plurality of features, each feature comprising a plurality of support-immobilized oligonucleotides hybridizable to at least a portion of a bisulfate converted genomic nucleic acid sample of a target organism, each oligonucleotide comprising, at each position of a dinucleotide pair complementary to a cytosine-guanine dinucleotide pair in an unconverted genomic nucleic acid of the target organism, a wobble base at each cytosine-complementary position.

11. The array of claim 10, wherein the wobble base is incorporated during oligonucleotide synthesis using an equimolar mixture of dNTPs.

12. The array of claim 11, wherein the equimolar mixture of dNTPs comprises deoxycytidine triphosphate (dCTP) and deoxythymidine triphosphate (dTTP).

13. The array of claim 12, wherein the equimolar mixture of dNTPs further comprises deoxyadenosine triphosphate (dATP), deoxyguanosine triphosphate (dGTP), or deoxyuridine triphosphate (dUTP).

14. The array of claim 10, wherein each oligonucleotide further comprises an adapter sequence at either or both ends of the oligonucleotide.

15. The array of claim 14, wherein the adapter sequence at either or both ends of the oligonucleotide comprises biotin.

16. The array of claim 14, wherein the adapter sequence at either or both ends of the oligonucleotide comprises a fluorophore.

17. The array of claim 10, wherein the oligonucleotides are capable of identifying a methylated base of a dinucleotide pair complementary to a cytosine-guanine dinucleotide pair in an unconverted genomic nucleic acid of the target organism.

18. The array of claim 10, wherein at least a subset of the oligonucleotides are capable of discriminating a thymine single nucleotide polymorphism (SNP) from an unmethylated cytosine in the unconverted genomic nucleic acid.

19. A method for identifying methylated bases within a bisulfite converted target nucleic acid sequence, the method comprising the steps of:
(a) contacting a plurality of oligonucleotides to a bisulfite converted nucleic acid sample, each oligonucleotide hybridizable to at least a portion of a bisulfite converted genomic nucleic acid sample of a target organism and each oligonucleotide comprising, at each position of a dinucleotide pair complementary to a cytosine-guanine dinucleotide pair in an unconverted genomic nucleic acid of the target organism, a wobble base at each cytosine-complementary position, whereby the contacting captures bisulfite converted target nucleic acid molecules in hybridization complexes with at least a portion of the plurality of oligonucleotides;
(b) separating the hybridization complexes from unbound and non-specifically bound nucleic acid molecules;
(c) eluting captured bisulfite converted target nucleic acid molecules from the hybridization complexes;
(d) sequencing eluted bisulfite converted target nucleic acid sequences; and
(e) identifying methylated bases of an eluted bisulfite converted target nucleic acid sequence, wherein identifying comprises comparing the unconverted genomic nucleic acid of the target organism to the eluted bisulfite converted target nucleic acid sequence, wherein a cytosine of the unconverted genomic nucleic acid is identified as unmethylated if the corresponding position in the eluted bisulfite converted target nucleic acid sequence is thymine, and wherein a cytosine of the unconverted genomic nucleic acid is identified as methylated if the corresponding position in the eluted bisulfite converted target nucleic acid sequence is cytosine.

20. The method of claim 19, wherein the wobble base is incorporated during oligonucleotide synthesis using an equimolar mixture of dNTPs.

21. The method of claim 20, wherein the equimolar mixture of dNTPs comprises deoxycytidine triphosphate (dCTP) and deoxythymidine triphosphate (dTTP).

22. The method of claim 21, wherein the equimolar mixture of dNTPs further comprises deoxyadenosine triphosphate (dATP), deoxyguanosine triphosphate (dGTP), or deoxyuridine triphosphate (dUTP).

23. The method of claim 19, further comprising amplifying the eluted bisulfite converted target nucleic acid sequences by polymerase chain reaction.

24. The method of claim 19, wherein the target nucleic acid sequence is genomic DNA.

25. The method of claim 19, wherein contacting occurs in the presence of bisulfite converted C0t1 DNA.

26. The method of claim 25, wherein the bisulfite converted C0t1 DNA and the target organism are of the same species.

27. The method of claim 19, wherein each oligonucleotide further comprises an adapter sequence at either or both ends of the oligonucleotide.

28. The method of claim 27, wherein the adapter sequence at either or both ends of the oligonucleotide comprises biotin.

29. The method of claim 27, wherein the adapter sequence at either or both ends of the oligonucleotide comprises a fluorophore.

30. The method of claim 19, wherein the oligonucleotides are support-immobilized.

31. The method of claim 19, further comprising discriminating a thymine single nucleotide polymorphism (SNP) from an unmethylated cytosine in the unconverted genomic nucleic acid.
